(19) United States
(12) Patent Application Publication
(10) Pub. No.: US 2021/0151178 A1
(43) Pub. Date: May 20, 2021

(54) SYSTEM, APPARATUS AND METHOD FOR MONITORING CLINICAL STATE OF A USER

(71) Applicant: INICU MEDICAL PRIVATE LIMITED, New Delhi (IN)

(72) Inventors: Harpreet SINGH, New Delhi (IN); Ravneet KAUR, New Delhi (IN); Suneyna BANSAL, Greater Noida UP (IN); Satish SALUJA, New Delhi (IN); Samir K. BRAHMACHARI, New Delhi (IN)

(21) Appl. No.: 16/634,057

(22) PCT Filed: Jul. 26, 2018
(86) PCT No.: PCT/IN2018/050491

§ 371 Date: Jan. 24, 2020

(30) Foreign Application Priority Data
Jul. 26, 2017 (IN) 201711026556

(51) Int. Cl. G16H 40/67; G16H 10/60; G16H 50/70; G16H 50/20; G16H 15/00; G16H 20/40; A61B 5/00; A61B 5/0205

(52) U.S. Cl. CPC G16H 40/67; G16H 10/60; G16H 50/70; G16H 50/20; G16H 40/20; G16H 20/40; A61B 5/7425; A61B 5/0205; A61B 5/749; G16H 15/00

(57) ABSTRACT

A system, apparatus and method for monitoring clinical state of a user is disclosed. The method for monitoring clinical state of the user comprises the steps of acquiring clinical information of the user from a set of medical devices and non-clinical information of the user from a database, accumulating, on at least one memory device, said clinical and non-clinical information to form an accumulated dataset, analyzing said accumulated dataset based on any or a combination of a predictive model and a historical data comparison model, wherein said analysis of the accumulated dataset comprises assessment of clinical state of the user based on which longitudinal state of the user is predicted over a defined time interval, and displaying, by a display unit, any or a combination of the analyzed clinical information and the analyzed non-clinical information forming part of the accumulated dataset.

### FIELD OF INVENTION

The present disclosure relates to patient monitoring methods and systems. In particular, the present disclosure relates to apparatuses, systems and methods for monitoring clinical state of a user.

### BACKGROUND OF THE INVENTION

Intensive care units (ICUs) are the most sterile and sensitive region of the hospitals. ICUs are equipped with multiple devices such as ventilator, incubator, Electrocardiogram (ECG) monitor etc. which continuously record and display the vitals of the patient. Apart from sensors to measure the patient's medical parameters, each of the devices has an additional display unit, which makes their design more bulky. Multiple display units of medical equipment's capture ICU's room space. Additionally, each medical device produces millions of proprietary data points per day for each patient and stores them only for 72 hours. Thus, lot of crucial information that could help in clinical decision-making gets lost. Processing of this high frequency voluminous physiological data streams is still a big challenge but could yield significant insights to provide quality care to the patients.

These critical patients need to be monitored for various other medical parameters, which could be obtained from their body fluids such as, blood, urine etc. Detailed examination including imaging like X-Rays etc. needs to be ordered to the laboratory. Ordered investigations and reports are maintained in Laboratory Information Management System (LIMS) and mostly linked to the Hospital Information System (HIS)/Electronic Medical Records (EMR). However, caregivers cannot frequently access this information through hospital's central system. Thus, they carry laboratory test results manually while examining the patient at the bedside.

These patients require repetitive monitoring by nurses and doctors. Detailed clinical examination of every patient at each single visit of clinician has to be recorded and maintained. All the prescribed medications, ordered investigations and nutritional data have to be recorded very carefully by the practitioners on paper. Nurses then follow these orders and maintain hourly records. Manual data entry on paper invokes numerous errors and would be an additional workload on caregivers. Additionally, few specialized ICUs such as Neonatal Intensive care units (NICUs) also need to continuously monitor for changing nutritional, calorific and sodium balance demands of newborn. This requires difficult computation and is error prone.

Till date, standardized clinically approved protocols are not implemented in every ICU across country and carried as manual record in ICU. Also these protocols may vary based on the facility of the hospital. Providing evidence based quality indicators of various ICUs can enforce protocol adherence. Also, this could also help in building the best practice to ensure quality care.

Clinically approved predictive scores have been extensively used in international clinical practices and have been statistically validated in their population. But there is no such record maintenance and thus, these scores have not been validated on global cohorts.

Advancement in technology or introduction of artificial intelligence has now enabled the mining of millions of real-time device data points to get the clinically relevant patterns. Thus there is enough literature evidence using deep-learning based probabilistic models using real time longitudinal data and reported results were seen to correlate well with morbidity and mortality status of the patients. However, this cognitive intelligence in the hardware is still not utilized in healthcare for quality care of patients.

Thus, there is a need for a unified platform integrating data from various devices and linking clinical data, lab records, clinical scores which can be maintainable by ICU specific protocols. Such automation of ICU workflow by robust big data infrastructure could facilitate monitoring and storage of the physiological and clinical phenotype parameters at various timeframes, and also help in clinical markers discovery process.

Moreover through such automation, longitudinal data of child's health since their birth (from NICU) or any patient admitted to ICUs could be stored and easily shared to patients even after their discharge. This would help the clinicians and researchers to explore the long term effect of early intervention and in understanding the causal-effect relationships among various co-morbid diseases. Post-discharge appointments, regular check-ups and laboratory tests could also be easily managed by such systems. Presently, all these reports and records need to be manually maintained by the patients. Also, connecting with the clinicians for telephonic advice in case of urgency is still a difficult task.

### OBJECTS OF THE INVENTION

A general objective of the invention is to provide a multipurpose, bedside smart and compact unified platform with single display screen to bridge data from multiple dimensions (medical devices, clinical data, LIMS, PACS) into one interface.

Another objective of the invention is to provide cognitive intelligent application to assist clinicians in pattern finding and real time analytics for early identification of disease.

Yet another objective of the invention is to enforce clinical protocols adherence by collecting evidence on impact of these practices to the quality parameters of ICUs across the country.

Yet another objective of the invention is to enable remote monitoring of ICUs present in resource crunch settings at rural regions by experts.

Yet another object of the invention is to provide a method for capturing, processing, analyzing and storing and displaying data using the system of the present disclosure.

Yet another object of the invention is to provide a post-discharge health surveillance using a wearable device to capture the patient's vitals at home (e.g. socks with sensors to capture HR, RR etc.) which would link to their mobile interface to monitor real time vitals along with all medical history of the patient.

### SUMMARY OF THE INVENTION

Accordingly, the present disclosure provides single, compact, touch sensitive display screen, and a voice assisted user interface that could be mounted over the patient bed without occupying room space. Various medical devices can be connected using standardized ports. Specific Health Level 7 (HL7)/American Society for Testing and Materials (ASTM) adaptations are used to establish connection with each of the medical device. All waveform data along with processed data would be displayed for each patient. This eliminates the need of individual display units for each device.

Present system seamlessly links with LIMS, HIS/ENIR and Picture Archiving and Communication System (PACS). Thus, raw data from laboratory reports as well as Image data can be viewed in a single click. Separate clinical assessment modules were designed with related laboratory tests, vitals and interventions. Doctor can select for the event/diagnosis and can order the assessment, which is linked to lab-side, and also followed by nurses using the same display. Nutrition will also be computed in a single click by using doctor's input and calorie received can also be viewed daily. Based on clinician's order, infusion pumps can be regulated which is attached to our unified platform.

Clinically approved protocols can be viewed and followed using this screen. This automation will reinforce implementation of clinically approved protocols for treatment by constant reminders and alerts. Quality indicator parameters can be seen to monitor the quality of ICUs by hospital management and further improvements can be made.

This big data hub has all unstructured and structured data coming from clinical, lab, physiological streams stored longitudinally for each patient. Thus, clinically tested scores and deep learning based analytical model utilizing real time longitudinal records are the part of the cognitive intelligence module. This accumulation of clinical data would provide cognitive assistance to reduce clinician difficulty in pattern recognition, which could help in clinical decision making to the practitioners in order to provide a more systematically approach for treatment of the patients.

Also, entire medical history (since ICU) would be anytime accessible on the patient's mobile application. This interface will give notifications/reminders/alerts such as immunization and follow up reminders. This would also serve as a transparent doctor-patient engagement platform. This application is connected to the wearable device e.g. socks with sensors to capture real-time vitals (Heart Rate (HR), Respiratory Rate (RR), etc.) at home that could be easily viewed by clinicians and patients. Such invention would not only help in easy monitoring and providing required care to the patient's even at home but would also ensure rapid intervention without any delay.

An aspect of the present disclosure relates to an apparatus for monitoring clinical state of a user, the apparatus including an information acquisition unit configured to acquire clinical information of the user from a set of medical devices and non-clinical information of the user from a database including historical vital information of the user, at least one memory device for collecting and storing said clinical and non-clinical information acquired from the set of medical devices and the database to form an accumulated dataset of the clinical and non-clinical information of the user, an information processing unit configured to analyze said accumulated dataset based on any or a combination of a predictive model and a historical data comparison model, wherein said analysis of the accumulated dataset includes assessment of clinical state of the user based on which longitudinal state of the user is predicted over a defined time interval, and a display unit adapted to display any or a combination of the analyzed clinical information and the analyzed non-clinical information forming part of the accumulated dataset.

In an embodiment, the apparatus further includes an information transmission unit configured to transmit any or a combination of the critical information and the non-critical information forming part of the accumulated dataset to a server/database.

In an embodiment, the information acquisition unit includes a communication interface configured to receive inputs from a user. In an embodiment, the communication interface is selected from a group consisting of an image capturing device and a user interface. In an embodiment, the user interface is any or a combination of a touch enabled screen and a voice enabled user interface.

Another aspect of the present disclosure relates to a method for monitoring clinical state of a user, the method including the steps of (i) acquiring clinical information of the user from a set of medical devices and non-clinical information of the user from a database including historical vital information of the user, (ii) accumulating, on at least one memory device, said clinical and non-clinical information acquired from the set of medical devices and the database to form an accumulated dataset of the clinical and non-clinical information of the user, (iii) analyzing said accumulated dataset based on any or a combination of a predictive model and a historical data comparison model, wherein said analysis of the accumulated dataset includes assessment of clinical state of the user based on which longitudinal state of the user is predicted over a defined time interval, and (iv) displaying, by a display unit, any or a combination of the analyzed clinical information and the analyzed non-clinical information forming part of the accumulated dataset.

In an embodiment, the method further includes a step of controlling one or more clinical parameters of the user, based on said prediction, by controlling one or more devices associated with said control of the one or more clinical parameters.

Another aspect of the present disclosure relates to a system for monitoring clinical state of a user. The proposed system includes a non-transitory storage device having embodied therein one or more routines operable to monitor clinical state of the user, and one or more processors coupled to the non-transitory storage device and operable to execute the one or more routines, wherein the one or more routines are executed in conjunction with a second set of routines stored on an application development and test server, and wherein the one or more routines include an information acquisition module, which when executed by the one or more processors, acquires clinical information of the user from a set of medical devices and non-clinical information of the user from a database including historical vital information of the user, an information storage module, which when executed by the one or more processors, collects and stores said clinical and non-clinical information acquired from the set of medical devices and the database to form an accumulated dataset of the clinical and non-clinical information of the user, an information processing module, which when executed by the one or more processors, analyzes said accumulated dataset based on any or a combination of a predictive model and a historical data comparison model, wherein said analysis of the accumulated dataset includes assessment of clinical state of the user based on which longitudinal state of the user is predicted over a defined time interval, and a display module, which when executed by the one or more processors, displays any or a combination of the analyzed clinical information and the analyzed non-clinical information forming part of the accumulated dataset.

In an embodiment, the system further includes an information transmission module configured to transmit any or a combination of the critical information and the non-critical information forming part of the accumulated dataset to a server/database.

In an embodiment, the set of medical devices incorporates any or a combination of cardio-respiratory monitors, pulse oximeters, blood gas machine and ventilators. In an embodiment, the medical devices are interfaced with any or a combination of Laboratory Information Management System (LIMS), Picture Archiving and Communication System (PACS), Hospital Information System (HIS) and Electronic Medical Record (EMR).

In an embodiment, based on said prediction of longitudinal state of the user over the defined time interval, the information processing unit enables control of one or more clinical parameters of the user by controlling one or more devices associated with said control of the one or more clinical parameters.

In an embodiment, the information acquisition module includes a communication interface configured to receive inputs from a user. In an embodiment, the communication interface is selected from a group consisting of an image capturing device and a user interface.

### BRIEF DESCRIPTION OF THE DRAWINGS

The accompanying drawings constitute a part of the description and are used to provide a further understanding of the present disclosure.

FIG. 1 illustrates an exemplary representation of various functional units/components of an apparatus that monitors clinical state of a user, in accordance with an embodiment of the present disclosure;

FIGS. 2A and 2B illustrate exemplary representations of a network architecture of proposed system for monitoring clinical state of the user, in accordance with an embodiment of the present disclosure;

FIG. 3 illustrates an exemplary representation of various functional modules of the proposed system, in accordance with an embodiment of the present disclosure;

FIG. 4 illustrates an exemplary flowchart representation of proposed method for monitoring clinical state of the user, in accordance with an embodiment of the present disclosure;

FIG. 5 illustrates an exemplary flowchart representation of various steps involved in the method for patient monitoring using the system and user interface in accordance with an embodiment of the present disclosure;

FIG. 6A illustrates the pictorial view of front panel displaying multiple tabs for concurrent function of multiple modules, in accordance with an embodiment of the present disclosure; and

FIG. 6B illustrates a rear panel displaying multiple ports to establish connections with devices and power supply, in accordance with an embodiment of the present disclosure.

### DETAILED DESCRIPTION OF THE INVENTION

It should be understood at the outset that although illustrative implementations of one or more embodiments of the present disclosure are provided below, the disclosed systems and/or methods may be implemented using any number of techniques, whether currently known or in existence. The disclosure should in no way be limited to the illustrative implementations, drawings, and techniques illustrated below, including the exemplary designs and implementations illustrated and described herein, but may be modified within the scope of the appended claims along with their full scope of equivalents.

The present invention pertains to an apparatus, system and method for monitoring clinical state of a user (also referred to as a “patient” hereinafter). More specifically, the present invention envisages to provide a unified platform integrating data from various devices and linking clinical data, lab records, clinical scores which can be maintainable by Intense care unit (ICU) specific protocols. Such automation of ICU workflow by robust big data infrastructure could facilitate monitoring and storage of the physiological and clinical phenotype parameters at various timeframes, and also help in clinical markers discovery process.

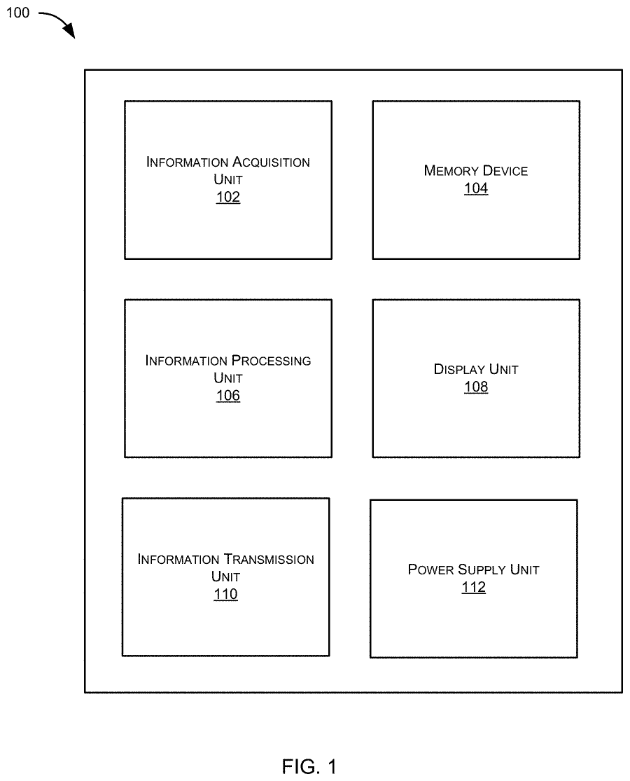

FIG. 1 illustrates an exemplary representation of various functional units/components of an apparatus that monitors clinical state of a user, in accordance with an embodiment of the present disclosure. The proposed apparatus (also referred to as “NEO box” hereinafter) 100 includes an information acquisition unit 102 that is adapted/configured to acquire clinical information of the user from a set of medical devices and non-clinical information of the user from a database including historical vital information of the user.

The apparatus 100 further includes at least one memory device 104 for collecting and storing said clinical and non-clinical information acquired from the set of medical devices and the database to form an accumulated dataset of the clinical and non-clinical information of the user. In an embodiment, the apparatus 100 can be interfaced/configured with a system in communication with the database to retrieve the requisite information.

In an embodiment, medical devices are connected to the proposed apparatus 100 through RJ45/RS232/USB connectors. Two RS232 ports are provisioned onto the main PCB whereas four RJ45 ports are designed on other small PCB which is named as RJ-45 PCB. 2 USB ports are available using an USB Hub to connect to WiFi using TP-link modem.

The apparatus 100 further includes an information processing unit 106 configured to analyze said accumulated dataset based on any or a combination of a predictive model and a historical data comparison model.

In an aspect, the analysis of the accumulated dataset includes assessment of clinical state of the user based on which longitudinal state of the user is predicted over a defined time interval.

The apparatus 100 further includes a display unit 108 adapted to display any or a combination of the analyzed clinical information and the analyzed non-clinical information forming part of the accumulated dataset. In an embodiment, the display unit 108 may notify and/or alert the user in case of any mismatch in transmission of real-time data to and from the system interfaced with the apparatus 100.

In an embodiment, the apparatus may further include an information transmission unit 110 configured to transmit any or a combination of the critical information and the non-critical information forming part of the accumulated dataset to a server/database.

In an embodiment, the information acquisition unit 102 includes a communication interface for receiving inputs from a user. The communication interface is selected from a group consisting of an image capturing device and an interactive touch interface. For instance, the communication interface may include two USB ports attached to the main PCB are used for programming the two BeagleBone.

In an embodiment, the power supply unit 112 may include an energy accumulator having any or a combination of nickel-cadmium (Ni—Cd) batteries, lithium ion batteries and ultra-capacitors to power various components/units of the apparatus 100, The power supply unit 112 acts as a standby power supply for the apparatus 100 in case when AC/DC power supply is not available, for example, during transportation of bed of a patient from one place to another to which the apparatus 100 is coupled/configured to.

In an embodiment, the apparatus 100 further comprises ports for power supply unit 112 and Ethernet and Internet connection. In an embodiment, power supply unit 112 of the proposed system allows the BeagleBone inside the proposed apparatus to run on Linux and shutdown properly when not in use. A power switch PCB is configured thereto to accomplish this by pulling down shutdown pin of the BBB initiating a soft shutdown.

Apart from medical devices, an IP camera is also interfaced with the NEO box 100, the IP camera being located bedside onto top of the patient using a flexible metallic connector that can be removed by clinician on discretion. This camera is integrated with NEO box 100 using RTSP on the local network to provide live feeds within the platform to the users/medical practitioners.

In an embodiment, the NEO box 100 is a neonatal bed side safety surveillance device that links with diverse bed side machines to acquire and store real-time data provided by them. The NEO box 100 also sends data to the cloud based clinical decision support system (interchangeably referred to as Integrated Neonatal Care Unit or iNICU hereinafter) linked with electronic medical records and lab information management system. Such integration ensures usage of medical devices, such as, cardio-respiratory monitors, pulse oximeters, blood gas machine and ventilators and the likes, as per the recommendation followed in clinical practice by sending alarms/notifications in case of any mismatch in equipment parameters with clinical orders. Thus, it would act as safety net by facilitating effective and seamless communications among biomedical devices and clinical management workflow. Additionally, availability of physiological time series data streams with other clinical and laboratory based observations enable us to utilize this data in cognitive intelligence module, which assists clinicians in timely clinical interventions. This data is also linked to the post discharge platform. This further enables longitudinal studies on high-risk neonates to study the long term implications of NICU interventions on newborn health.

In an embodiment, setting up the NEO box 100 in the NICU requires programming the BBB using USB programming interface. The complete “from scratch” setup of the BBB involves installation of Java 1.8, RXTX library and enabling of tty01 port for serial-RS-232 communication. Ethernet network and WiFi (TPLink) setup is achieved using connman device drivers available in Debian operating system. Bulldog API is used for programming GPIO pins on beaglebone to light LED's. On system start, two crontabs are scheduled: the first establishes the network state of BeagleBone so that it can connect to devices with the correct network settings and second crontab calls the java program i.e. jar to invoke the software program from shell file.

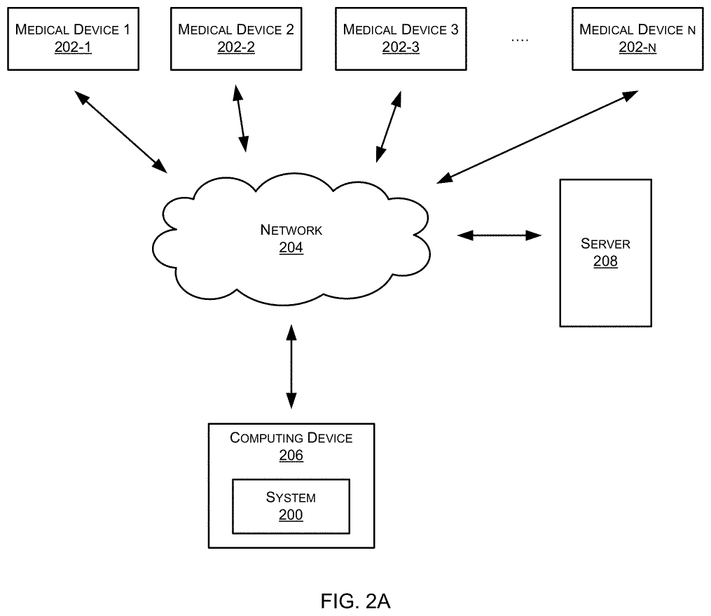

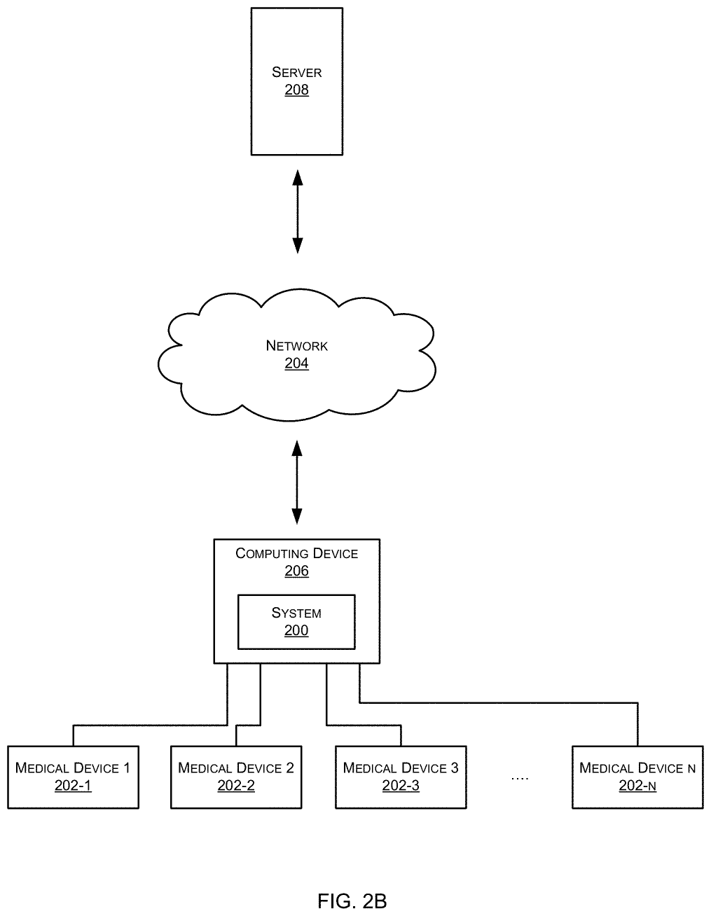

FIGS. 2A and 2B illustrate exemplary representations of a network architecture of proposed system for monitoring clinical state of the user, in accordance with an embodiment of the present disclosure.

In an aspect, the proposed system 200 can be configured with a computing device 206 interfaced with Internet of Things (IoT) devices/appliances. The system 200 may also be in communication with a server/server 208 that incorporates various analytical and statistical tools, for instance, Apache Cassandra database, to facilitate analysis of clinical as well as non-clinical information of the user. The system 200 can communicate with a plurality of medical devices/systems 202-1, 202-2, 202-3, . . . 202-N (also individually referred to as “medical device 202” and collectively referred to as “medical devices 202” hereinafter), such as, but not limited to, cardio-respiratory monitors, pulse oximeters, blood gas machine and ventilators via a network 204 to perform the intended task of monitoring clinical status of the user. In an embodiment, the proposed system 200 may reside in any of the computing device 206 or the server 208. In an embodiment, the computing device 206 may be any of a proprietary apparatus (as illustrated in FIG. 1), a mobile device, a laptop, a personal computer, a personal digital assistant (PDA), a network device, and the likes. In an embodiment, the proposed system 200 is implemented with the proposed apparatus 100.

In an embodiment, the medical devices are interfaced with any or a combination of Laboratory Information Management System (LIMS), Picture Archiving and Communication System (PACS), Hospital Information System (HIS) and Electronic Medical Record (EMR).

The network 204 may allow the system 200 to communicate with the server 208 and other peripherals via any one or more of, for instance, a local intranet, a Personal Area Network (PAN), a Local Area Network (LAN), a Wide Area Network (WAN), a Virtual Private Network (VPN), an Advanced Intelligent Network (AIN) connection, a Synchronous Optical Network (SONET) connection, a digital line connection, Digital Data Service (DDS) connection, DSL (Digital Subscriber Line) connection, an Ethernet connection, an ISDN (Integrated Services Digital Network) line or a dial-up port a cable modem. Further, communications may also include links to any of a variety of wireless networks, including Wireless Application Protocol (WAP), General Packet Radio Service (GPRS), Global System for Mobile Communication (GSM), Code Division Multiple Access (CDMA) or Time Division Multiple Access (TDMA), cellular phone networks, Bluetooth radio, or an IEEE 802.11-based radio frequency network. The network 204 can further include or interface with any one or more of an RS-232 serial connection, an IEEE-1394 connection, a Fiber Channel connection, an IrDA (infrared) port, a SCSI (Small Computer Systems Interface) connection, a USB (Universal Serial Bus) connection or other wired or wireless, digital or analog interface or connection, mesh or other networking. In an embodiment, the network is a Big Data interface that manages workflow of the proposed system 200 and transmits real-time data to and from the system 200.

In an aspect, the system 200 can acquire clinical information of the user from a set of medical devices and non-clinical information of the user from a database including historical vital information of the user. The system 200 can collect and store said clinical and non-clinical information acquired from the set of medical devices and the database to form an accumulated dataset of the clinical and non-clinical information of the user. In an embodiment, medical devices 200 may be connected to the proposed system 200 through the network 204.

Referring now to FIG. 2B, the medical devices 202 may be connected to the proposed system 200 through RJ45/RS232/USB connectors of the computing device 206, where the computing device 206 is a proprietary apparatus (NEO box) 100, as illustrated in FIG. 1. Two RS232 ports are provisioned onto the main PCB of the computing device 206 whereas four RJ45 ports are designed on other small PCB of the computing device 206 which is named as RJ-45 PCB. 2 USB ports are available using an USB Hub to connect to WiFi using TP-link modem.

In an embodiment, during data acquisition various if conditions are implemented in a thread to acquire data, which runs every minute. The information acquisition module 302 first receives data from a medical device, parses and then sends it into dataSend (key/value pair). For this, there is a separate thread that runs every minute which sends data. Then data is picked from dataSend and convert into JSON format. Now, this JSON is sent to cloud (iNICU) using wi-fi through TPLink. Each acquisition is a daemon (independent thread and each send is an independent thread). Finally, information acquisition module 302 receives acknowledgement from server and finishes one thread of data acquisition. This loop is then repeated every minute.

In an aspect, the system 200 can analyze said accumulated dataset based on any or a combination of a predictive model and a historical data comparison model, wherein said analysis of the accumulated dataset includes assessment of clinical state of the user based on which longitudinal state of the user is predicted over a defined time interval, and display any or a combination of the analyzed clinical information and the analyzed non-clinical information forming part of the accumulated dataset. In an embodiment, the display unit may include four LEDs to indicate different states of the system 200. One LED displays whether a BeagleBone Black device (BBB) configured/connected thereto is functional and java program incorporated therein is running or not. A second LED is turned on when the apparatus 100 is receiving data from the medical devices. A third LED lights up if a TPLink is sending data to the database and a fourth LED blinks when the apparatus 100 receives acknowledgment from the database.

The proposed system 200 aggregates different parameters of different medical devices to allow linking of their physiological, clinical as well as laboratory data over time in order to correlate various disease conditions. This physiological time series data linked with disease conditions enables correlation of proper usage of device in given diseases state or its association with a later episode witnessed during patient stay in the ICU.

In an embodiment, the system 200 may transmit any or a combination of the critical information and the non-critical information forming part of the accumulated dataset to a server/database for storage or for further processing.

It will be understood that the functionalities described herein, which are attributed to the system 200 may also be executed within the computing device 206. That is, the computing device 206 may be programmed to execute the functionalities described herein. In other instances, the system 200 and computing device 206 may cooperate to provide the functionalities described herein, such that the computing device 206 is provided with a client-side application that interacts with the system 200 such that the system 200 and computing device 206 operate in a client/server relationship.

Neonatal intensive care unit (NICU) is one such ICU specialized in care of ill and preterm newborns which can be tremendously benefited by adopting such EHR system built on its domain specific ontology. The NICU is a complex system where many factors have been found to influence the intrinsic risk of medical errors, such as heavy workload, poor staff strength, poor communication among team member and inadequate knowledge & training.

In an embodiment, the proposed system 200 may be interfaced with a NICU to standardize neonatal care, thereby enabling comparative measurement of quality parameters across units through the common domain ontology. This enables collection and comparison of clinical data in a consistent way from different NICUs.

The proposed system 200 can also utilize Integrated Child Health Record (iCHR) to capture longitudinal data till 12 years of age for phenotypic, molecular and clinical markers discovery process.

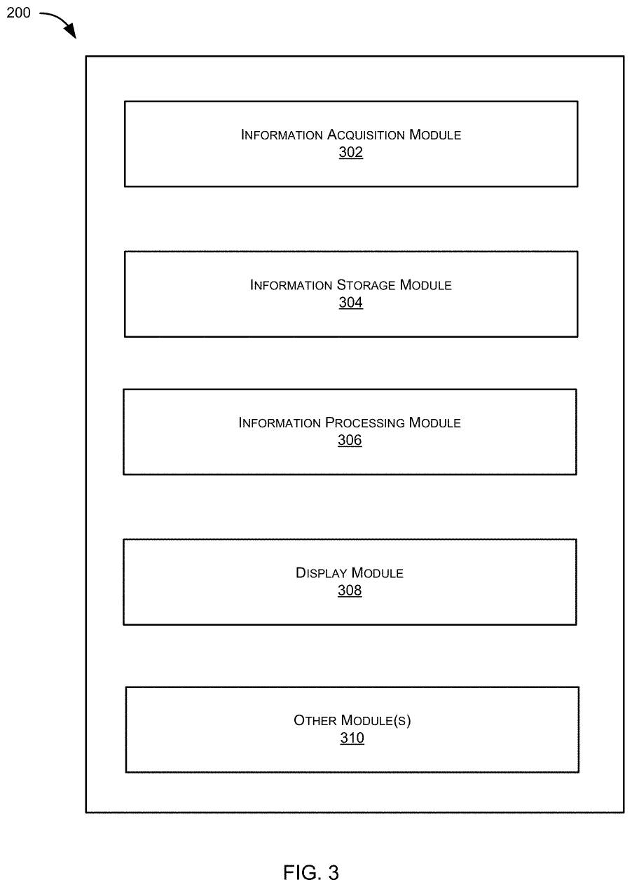

FIG. 3 illustrates an exemplary block diagram of the functional modules of the proposed system, showing the main functional modules and the functional module interconnections. Typical hardware and electronic components are arranged to perform the said intended task of monitoring clinical state of the user.

The system 200 as illustrated in FIGS. 2A and 2B, generally comprises one or more processors, a network interface, and a memory. The memory comprises logic (e.g., instructions) that can be executed by the processor to perform various methods, which are described in greater detail herein. The memory may store various functions modules that are executable by the one or more processors, the functional modules including, an information acquisition module 302, an information storage module 304, an information processing module 306, a display module 308 and other module(s) 310 that are necessary to carry out the intended function of monitoring clinical state of the user/patient.

In an aspect, the information acquisition module 302 acquires clinical information of the user from a set of medical devices and non-clinical information of the user from a database including historical vital information of the user.

In an embodiment, the information acquisition module 302 consists of two programmable BeagleBone black microprocessor running Java 1.8 on Debian operating system. Each BeagleBone (BBB) has 1 USB, 1 RJ45 and RS232 interface serial pins to acquire data coming from devices and camera, respectively. One USB port is configured to program the beagle bone from the computing device.

In an embodiment, medical devices 200 may be connected to the proposed system 200 through the network 204. In another embodiment, the medical devices 202 may be connected to the proposed system 200 through RJ45/RS232/USB connectors of the computing device 206, where the computing device 206 is a proprietary apparatus (NEO box) 100, as illustrated in FIG. 1. Two RS232 ports are provisioned onto the main PCB of the computing device 206 whereas four RJ45 ports are designed on other small PCB of the computing device 206 which is named as RJ-45 PCB. 2 USB ports are available using an USB Hub to connect to WiFi using TP-link modem.

In an aspect, the information storage module 304 collects and stores said clinical and non-clinical information acquired from the set of medical devices and the database to form an accumulated dataset of the clinical and non-clinical information of the user.

In an aspect, the information processing module 306 analyzes said accumulated dataset based on any or a combination of a predictive model and a historical data comparison model. In an embodiment, the analysis of the accumulated dataset includes assessment of clinical state of the user based on which longitudinal state of the user is predicted over a defined time interval.

In an embodiment, based on said prediction of longitudinal state of the user over the defined time interval, the information processing unit enables control of one or more clinical parameters of the user by controlling one or more devices associated with said control of the one or more clinical parameters.

In an aspect, the display module 308 displays any or a combination of the analyzed clinical information and the analyzed non-clinical information forming part of the accumulated dataset. In an embodiment, the display module 308 may notify and/or alert the user in case of any mismatch in transmission of real-time data to and from the system 200.

In an embodiment, the display module 308 may include four LEDs to indicate different states of the system 200. One LED displays whether a BBB configured/connected thereto is functional and java program incorporated therein is running or not. A second LED is turned on when the system 200 is receiving data from the medical devices. A third LED lights up if a modem, say TPLink, is sending data to the database and a fourth LED blinks when the system 200 receives acknowledgment from the database.

In an embodiment, the other module(s) 310 may include an information transmission module configured to transmit any or a combination of the critical information and the non-critical information forming part of the accumulated dataset to a server. In an embodiment, the information transmission module consists of a TPLink WiFi USB dongle to send acquired data from BeagleBone to the server/cloud/database. TP-link can be connected using the USB Hub PCB, which internally gets connected, to USB port at BBB.

In an embodiment, the information acquisition module 302 includes a communication interface configured to receive inputs from a user. In an embodiment, the communication interface is selected from a group consisting of an image capturing device and an interactive touch interface. For instance, the communication interface may include two USB ports attached to the main PCB are used for programming the two BeagleBone. In an embodiment, the system 200 further comprises ports for power supply and Ethernet and Internet connection. In an embodiment, the power supply of the proposed system allows the BeagleBone inside the proposed apparatus to run on Linux and shutdown properly when not in use. A power switch PCB is configured thereto to accomplish this by pulling down shutdown pin of the BBB initiating a soft shutdown.

In an embodiment, device data from various medical devices can be fetched by the information acquisition module 302. Device vendors may support Health Level Protocol (HL7) for data retrieval from their devices. The system 200 uses open source HAPI (I-1L7 API) and supports HL7 2.X version to fetch data from various medical devices in ICU settings. Separate thread is initiated for each supporting device to acquire concurrent live data feeds. Various device specific HL7 adaptations are used to establish connection with each of the medical device.

Medical device data integration (MDDI) layer is divided into two blocks, i.e., client and server. Client code is running on BBB which provides wrapper on HL7 and RS 232 allowing data acquisition from various devices. Client side code uses open source HAPI (HL7 API) that supports HL7 2.X and 3.X version to fetch data from various devices in NICU settings. For RS 232 interface, RXTX Java based communication API is used on BBB. Separate thread is initiated for each supporting medical device to acquire concurrent live data feeds. Java codes running on BBB acquires data from device and parse it in JSON format. Parsed data is then transmitted to server layer along with necessary parameters, Patient id (or UHID), apparatus identifier, mac id of the apparatus and medical device name. The apparatus name is the name of the iNICU medical device/apparatus that would be provided to the NICU facility. Server layer is implemented using open source Spring-Boot and Apache Cassandra. Server subscribes to real-time streams of unstructured data coming from various client implementations and stores it in NoSQL Cassandra database. Only if the received data has a valid mapping with an active UHID, data gets stored into the Cassandra database. Server software platform is also integrated with LIMS via ASTM protocol (JAVA ASTM API). Image capturing device is built using OpenCV Python library. Real-Time Streaming Protocol (RTSP) is used to establish connection with the image capturing device placed on patient bedside. Real-time image frames of patients in .jpg format are encrypted, parsed in JSON format and sent to MDDI server layer along with the apparatus name.

For RS 232 USB interface, RXTX Java based communication API (Application Program Interface) is used. Data aggregation from these devices is carried out using Internet of things (IoT) gateway.

Machine Data Integration (MDI) Server layer is implemented using open source Apache Kafka. MDI Server subscribes to real time streams of data coming from various MDI client implementations. It uses Apache Cassandra to store unstructured data. MDI Server piece also integrated with HMS via ASTM protocol (JAVA ASTM API).

In an embodiment, the interactive user interface is built using service oriented architecture. The user interface may include any or a combination of touch enabled screens or an Artificial Intelligence (AI) voice assisted/guidance system, for example, Amazon Alexa™, Microsoft Cortana™ and the like voice assistance systems. The server part is implemented using Java 1.8 language leveraging Spring Boot framework. User Interface layer is built using responsive AngularJS (JavaScript Framework) and HTML5. This allows User Interface layer to be responsive and it can run seamlessly on Tablet, Laptop and Mobile devices. JSON based REST API integration connects AngularJS and Spring layers. Patient data is accessed either from Hospital Information System as Admission Discharge Transfer (ADT) events through HL7 Integration or manually entered by the Hospital administration. Clinical data is automatically and frequently stored in PostgreSQL and Hibernate allows access of database from Java business layer. Various Neonatal calculators are coded using Drools rule engine and stored as metadata. Solution is hosted on IBM Softlayer based cloud infrastructure. Growth charts are implemented using high charts and JavaScript. Cloud component allows only HTTPS based communication protected by 256-bit encryption with web interface.

In an exemplary implementation, continuous data stream from various medical devices provide, for instance, 39 fields present in the iNICU data dictionary. Cassandra database stores 11 such fields from cardio-respiratory monitors, 14 from ventilators and 14 from blood gas. Raw data persisted in Cassandra database with keyspace ‘inicu’ and table named as ‘inicu.patient continuous’. Compaction strategy used is based on the time window. Thus, time series data segregated on various clusters using time as primary key. Cluster keys are UHID, minute, hour and day. Supplementary Excel 1 contains continuous data coming from monitors and ventilators for a subset of 40 de-identified newborns. Raw data points from Cassandra database is then processed to store every minute value in PostgreSQL which is available to the end user at iNICU interface.

In an embodiment, the clinical information and the non-clinical information acquired by the information acquisition module 302 is stored in PostgreSQL and waveform/machine data is stored in Apache Cassandra. Normalized data is fetched from both unstructured and structured data stores. Disease based neonatal score helps to categorize infant into different diseases. Incoming facts (urine output, RR, HR, peripheral capillary oxygen saturation (SpO₂) etc.) of child act as input to Clinical Rules and matching rules inferences are executed. These inferences generate alarm and notification which are sent via SMS/Google Cloud Messaging and Apple Push Notification Service to doctor, nurses and patients (specific one).

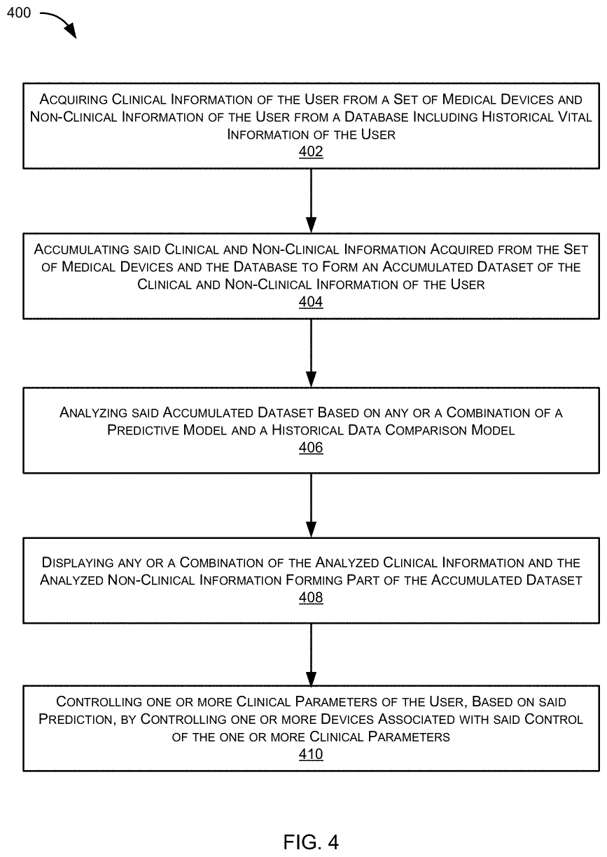

FIG. 4 illustrates an exemplary flowchart representation of proposed method for monitoring clinical state of the user, in accordance with an embodiment of the present disclosure. In an aspect, the proposed method 400 may include, at step 402, acquiring clinical information of the user from a set of medical devices and non-clinical information of the user from a database including historical vital information of the user.

The method 400 may include, at step 404, accumulating, on at least one memory device, said clinical and non-clinical information acquired from the set of medical devices and the database to form an accumulated dataset of the clinical and non-clinical information of the user.

The method 400 may further include, at step 406, analyzing said accumulated dataset based on any or a combination of a predictive model and a historical data comparison model, wherein said analysis of the accumulated dataset includes assessment of clinical state of the user based on which longitudinal state of the user is predicted over a defined time interval.

The method 400 may further include, at step 408, displaying, by a display unit, any or a combination of the analyzed clinical information and the analyzed non-clinical information forming part of the accumulated dataset.

In an embodiment, the method may further include a step 410 of controlling one or more clinical parameters of the user, based on said prediction, by controlling one or more devices associated with said control of the one or more clinical parameters.

It would be appreciated that the proposed method/process may be implemented as a hardware module and/or a software module. For example, method 400 can be implemented as application-specific circuitry or as a software module including instructions stored at a memory and executed at a processor in communication with the memory.

In an embodiment, mapping with the patient id is needed for the bed side devices like monitor and ventilator. Once the NEO box is connected with the monitor and/or ventilator and the probes are attached to the patient, a user needs to click Add device module at iNICU application from dashboard patient card to connect the right NEO box number. Also it is equally important to disconnect the NEO box from application interface in case the patient gets disconnected from the medical device which may happen in case of bed change, machine change, transfer to step down facility and discharge.

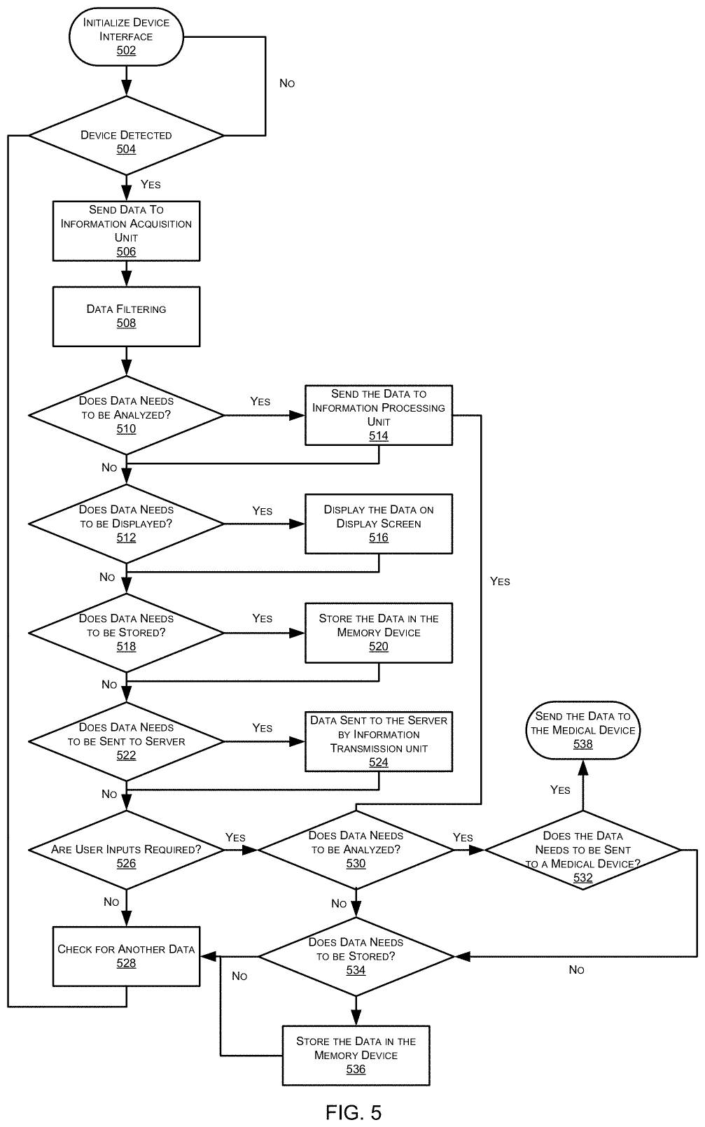

FIG. 5 illustrates an exemplary flowchart representation of various steps involved in the method for patient monitoring using the proposed system and apparatus in accordance with an embodiment of the present disclosure. In an embodiment, the method includes a step 502 of initiating user interface of the proposed apparatus/device, as shown in FIG. 1. Thereafter, at step 504, connection of the device with the proposed system is detected. If the device is not detected, then the system again initiates device interface to detect devices. If the device is detected, then, at step 506, data is sent to the data acquisition unit and at step 508, the data is filtered. The system then determines, at step 510, if the filtered data needs to be analyzed. If not, at step 512, the system checks whether the data is to be displayed or not. If required, at step 516, the data without analysis is displayed on a display screen of the display unit.

If at step 510, the system determines that the data needs to be analyzed, then at step 514, the data is sent to the data analysis processor, where the data is analyzed. After the analysis of data by the processor, the system checks again if the data needs to be displayed or not. If required, the analyzed data is displayed on the user interface screen: The system then determines, at step 518, if the data needs to be stored or not. If the data is required to be stored, at step 520, the data is stored in the memory device, or else at step 522, the system checks if the data is required to be sent to a server/cloud. If yes, at step 524, the data is sent to the cloud using internet or Ethernet connection.

The system then determines, at step 526, if any inputs are required from the user. If not, the system, at step 528, checks for another data, based on which device is detected and the process starts again. If at step 526, user inputs are required, then at step 530, the system determines whether the new data derived from user inputs needs to be analyzed. If the data is to be analyzed, the data is sent to the data processing unit. If at step 530, the data is not required to be analyzed, the system then checks, at step 532, if the data is required to be sent to the device to control it and at step 538, sends the required data to the device if the same is required; or at step 534, checks if the data needs to be stored. If the data is not required to be analyzed but stored or if the data is not required to be analyzed and not to be sent to the device for controlling but stored, then at step 536, the data is stored in the memory. If the data is not required to be stored at all, the system checks for another data and detects the device and the process continues.

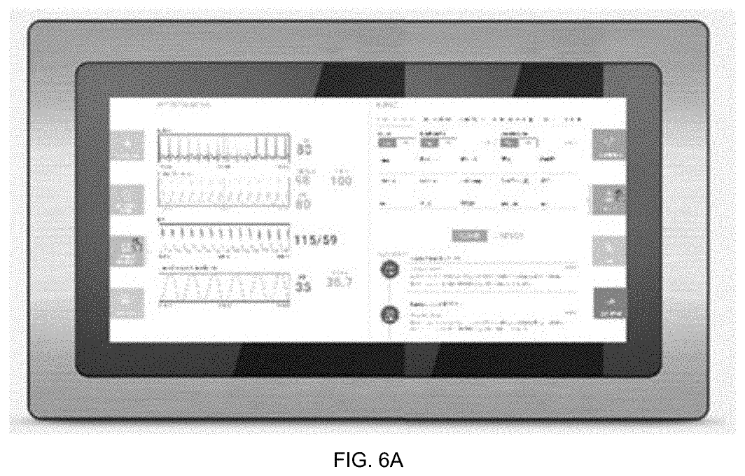

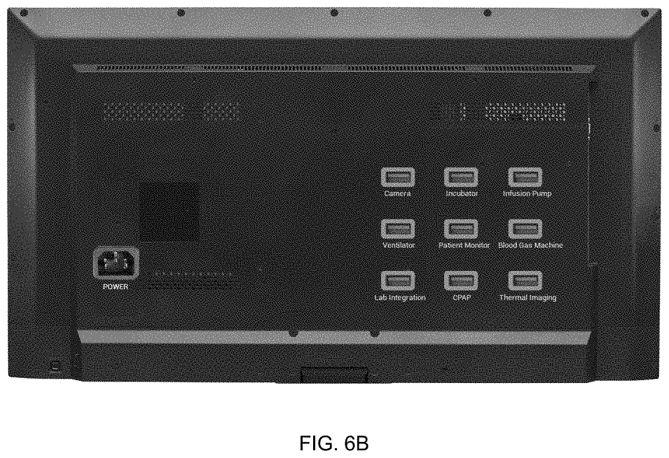

FIG. 6A illustrates the pictorial view of front panel displaying multiple tabs for concurrent function of multiple modules, in accordance with an embodiment of the present disclosure. An interactive touch interface according to an embodiment of the present invention is shown. Various medical devices can be connected to the monitoring system of the present disclosure and clinical data recorded by these devices can be displayed on the single interactive touch user interface. These devices can be regulated directly from the user defined input at the interactive touch user interface of the invention. User can also input for other relevant clinical information that needs to be stored in the memory and display in nursing modules as clinician's order.

In an embodiment, the iNICU interface displays concurrent data streams of physiological data coming from diverse devices onto a single screen which can be further interpreted by the clinicians for any correlation or cross-correlation among the parameters even remotely at any point of time. Moreover, data can be visualized and also downloaded as csv files at different sampling frequency (hourly or minute data points) for any kind of temporal analysis by users. Apart from trends of data, current values of these variables are visible at carious locations in a web application. A dashboard screen which displays the current status for all the patients admitted in the NICU unit at once. This screen provides a first glimpse of current state of all patients along with real-time device parameters admitted in NICU unit. Thus, the proposed NEO box facilitates in devising a clinical decision at first sight for the clinical deterioration of the patient who may needs immediate intervention without even entering the entire data of the patient into the system.

FIG. 6B illustrates a rear panel displaying multiple ports to establish connections with devices and power supply, in accordance with an embodiment of the present disclosure. Various medical devices could be connected through the R.147/RS232/USB connectors to the system such that the clinical as well as non-clinical data from these medical devices connected to the monitoring system is displayed on the single interactive touch user interface as shown in FIG. 6A.

Various modifications to these embodiments are apparent to those skilled in the art from the description and drawings herein. The principles associated with the various embodiment defined herein may be applied to other embodiments. Therefore, the description is not intended to be limited to the embodiments shown along with the accompanying drawings but is to be provided broadest scope consistent with the principles and novel and inventive features describe/disclosed or suggested herein. Any modifications, equivalent substitutions, improvements etc. within the spirit and principle of the present invention shall all be included in the scope of protection of the present invention.

### Advantages of the Invention

The present disclosure provides a multipurpose, bedside smart and compact unified platform with single display screen to bridge data from multiple dimensions (medical devices, clinical data, LIMS, PACS) into one interface.

The present disclosure provides a cognitive intelligent application to assist clinicians in pattern finding and real time analytics for early identification of disease.

The present disclosure envisages to effectively enforce clinical protocols adherence by collecting evidence on impact of these practices to the quality parameters of ICUs across the country.

The present disclosure envisages to enable remote monitoring of ICUs present in resource crunch settings at rural regions by experts.

The present disclosure provides a method for capturing, processing, analyzing and storing and displaying data using the system of the present disclosure.

The present disclosure provides a post-discharge health surveillance using a wearable device to capture the patient's vitals at home (e.g. socks with sensors to capture HR, RR etc.) which would link to their mobile interface to monitor real time vitals along with all medical history of the patient.

1. An apparatus for monitoring clinical state of a user, the apparatus comprising:
an information acquisition unit configured to acquire clinical information of the user from a set of medical devices and non-clinical information of the user from a database comprising historical vital information of the user;
at least one memory device for collecting and storing said clinical and non-clinical information acquired from the set of medical devices and the database to form an accumulated dataset of the clinical and non-clinical information of the user;
an information processing unit configured to analyze said accumulated dataset based on any or a combination of a predictive model and a historical data comparison model, wherein said analysis of the accumulated dataset comprises assessment of clinical state of the user based on which longitudinal state of the user is predicted over a defined time interval; and
a display unit adapted to display any or a combination of the analyzed clinical information and the analyzed non-clinical information forming part of the accumulated dataset.

2. The apparatus as claimed in claim 1, wherein the set of medical devices incorporates any or a combination of cardio-respiratory monitors, pulse oximeters, blood gas machine and ventilators.

3. The apparatus as claimed in claim 1, wherein based on said prediction of longitudinal state of the user over the defined time interval, the information processing unit enables control of one or more clinical parameters of the user by controlling one or more devices associated with said control of the one or more clinical parameters.

4. The apparatus as claimed in claim 1, further comprising an information transmission unit configured to transmit any or a combination of the critical information and the non-critical information forming part of the accumulated dataset to a server/database.

5. The apparatus as claimed in claim 1, wherein the information acquisition unit comprises a communication interface configured to receive inputs from a user, and wherein the communication interface is selected from a group consisting of an image capturing device and a user interface.

6. The apparatus as claimed in claim 5, wherein the communication interface is any or a combination of a touch enabled display screen and a voice assisted guidance system.

7. A method for monitoring clinical state of a user, the method comprising the steps of:
acquiring clinical information of the user from a set of medical devices and non-clinical information of the user from a database comprising historical vital information of the user;
accumulating, on at least one memory device, said clinical and non-clinical information acquired from the set of medical devices and the database to form an accumulated dataset of the clinical and non-clinical information of the user;
analyzing said accumulated dataset based on any or a combination of a predictive model and a historical data comparison model, wherein said analysis of the accumulated dataset comprises assessment of clinical state of the user based on which longitudinal state of the user is predicted over a defined time interval; and
displaying, by a display unit, any or a combination of the analyzed clinical information and the analyzed non-clinical information forming part of the accumulated dataset.

8. The method as claimed in claim 7, further comprising a step of controlling one or more clinical parameters of the user, based on said analysis, by controlling one or more devices associated with said control of the one or more clinical parameters.

9. The method as claimed in claim 8, wherein the one or more devices comprise any or a combination of cardio-respiratory monitors, pulse oximeters, blood gas machine and ventilators.

10. A system for monitoring clinical state of a user comprising:
a non-transitory storage device having embodied therein one or more routines operable to monitor clinical state of the user; and
one or more processors coupled to the non-transitory storage device and operable to execute the one or more routines, wherein the one or more routines are executed in conjunction with a second set of routines stored on an application development and test server, and wherein the one or more routines include:
an information acquisition module, which when executed by the one or more processors, acquires clinical information of the user from a set of medical devices and non-clinical information of the user from a database comprising historical vital information of the user;
an information storage module, which when executed by the one or more processors, collects and stores said clinical and non-clinical information acquired from the set of medical devices and the database to form an accumulated dataset of the clinical and non-clinical information of the user;
an information processing module, which when executed by the one or more processors, analyzes said accumulated dataset based on any or a combination of a predictive model and a historical data comparison model, wherein said analysis of the accumulated dataset comprises assessment of clinical state of the user based on which longitudinal state of the user is predicted over a defined time interval; and
a display module, which when executed by the one or more processors, displays any or a combination of the analyzed clinical information and the analyzed non-clinical information forming part of the accumulated dataset.

11. The system as claimed in claim 10, wherein the set of medical devices incorporates any or a combination of cardio-respiratory monitors, pulse oximeters, blood gas machine and ventilators.

12. The system as claimed in claim 10, wherein based on said prediction of longitudinal state of the user over the defined time interval, the information processing module enables control of one or more clinical parameters of the user by controlling one or more devices associated with said control of the one or more clinical parameters.

13. The system as claimed in claim 10, further comprising an information transmission module configured to transmit any or a combination of the critical information and the non-critical information forming part of the accumulated dataset to a server/database.

14. The system as claimed in claim 10, wherein the information acquisition module comprises a communication interface configured to receive inputs from a user, and wherein the communication interface is selected from a group consisting of an image capturing device and a user interface.

15. The system as claimed in claim 10, wherein the communication interface is any or a combination of a touch enabled display screen and a voice assisted guidance system.
